SEQUENCE LISTING (11 sequences)

SEQ ID NO: 1 - DNA, 243 bp, Homo sapiens
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgc

SEQ ID NO: 2 - DNA, 513 bp, Homo sapiens
ccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctcccc

SEQ ID NO: 3 - DNA, 243 bp, Artificial
  Synthetic EFS promoter in reverse orientation
gcgtcacgacacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcc

SEQ ID NO: 4 - DNA, 40 bp, Artificial
  Synthetic linker
ttaattaaacgcctaccctcgagtagcttgatatgctagc

SEQ ID NO: 5 - DNA, 796 bp, Artificial
  Synthetic bidirectional promoter
gcgtcacgacacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagccttaattaaacgcctaccctcgagtagcttgatatgctagcccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctcccc

SEQ ID NO: 6 - DNA, 816 bp, Artificial
  Synthetic deltaLNGFRco sequence
atggatggccctagactcctccttctcctgctgctgggcgtgtcactgggcggagccaaagaggcctgtcctaccggcctgtacacacacagcggcgagtgctgcaaggcctgcaatctgggagaaggcgtggcccagccttgcggcgctaatcagaccgtgtgcgagccctgcctggacagcgtgacctttagcgacgtggtgtccgccaccgagccttgcaagccttgtaccgagtgtgtgggcctgcagagcatgagcgccccttgcgtggaagccgacgatgccgtgtgcagatgcgcctacggctactaccaggacgagacaaccggcagatgcgaggcctgtagagtgtgcgaggccggatctggcctggtgttcagttgtcaagacaagcagaacaccgtgtgtgaagagtgccccgacggcacctacagcgacgaggccaatcacgtggacccctgcctgccatgcacagtgtgcgaagataccgagcggcagctgcgcgagtgtaccagatgggccgatgccgagtgcgaagagatccctggcagatggatcaccagaagcaccccccctgagggcagcgatagcacagcccctagcacccaggaacctgaggcccctcctgagcaggatctgatcgcctctacagtggccggcgtcgtgaccacagtgatgggcagttctcagcccgtcgtgacaagaggcaccaccgacaacctgatccccgtgtactgcagcatcctggccgctgtggtcgtgggcctggtggcctatatcgccttcaagcggtggaaccggggcatcctgtga

SEQ ID NO: 7 - DNA, 1296 bp, Artificial
  Synthetic hFoxp3co sequence (codon optimized)
atgcccaaccccagacccggaaagcctagcgccccttctctggccctgggaccttctcctggcgcctccccatcttggagagccgcccctaaagccagcgatctgctgggagctagaggccctggcggcacattccagggcagagatctgagaggcggagcccacgcctctagcagcagcctgaatcccatgccccctagccagctgcagctgcctacactgcctctcgtgatggtggcccctagcggagctagactgggccctctgcctcatctgcaggccctgctgcaggacagaccccacttcatgcaccagctgagcaccgtggatgcccacgccagaacacctgtgctgcaggtgcaccccctggaaagccctgccatgatcagcctgacccctccaaccacagccaccggcgtgttcagcctgaaggccagacctggactgccccctggcatcaatgtggccagcctggaatgggtgtcccgcgaacctgccctgctgtgcaccttccccaatcccagcgcccccagaaaggacagcacactgtctgccgtgccccagagcagctatcccctgctggctaacggcgtgtgcaagtggcctggctgcgagaaggtgttcgaggaacccgaggacttcctgaagcactgccaggccgaccatctgctggacgagaaaggcagagcccagtgtctgctgcagcgcgagatggtgcagagcctggaacagcagctggtgctggaaaaagaaaagctgagcgccatgcaggcccacctggccggaaaaatggccctgacaaaggccagcagcgtggccagctctgacaagggcagctgctgcattgtggccgctggctctcagggacctgtggtgcctgcttggagcggacctagagaggcccccgatagcctgtttgccgtgcggagacacctgtggggcagccacggcaactctaccttccccgagttcctgcacaacatggactacttcaagttccacaacatgaggccccccttcacctacgccaccctgatcagatgggccattctggaagcccccgagaagcagcggaccctgaacgagatctaccactggtttacccggatgttcgccttcttccggaaccaccccgccacctggaagaacgccatccggcacaatctgagcctgcacaagtgcttcgtgcgggtggaaagcgagaagggcgccgtgtggacagtggacgagctggaatttcggaagaagcggtcccagaggcccagccggtgtagcaatcctacccctggcccttga

SEQ ID NO: 8 - DNA, 816 bp, Artificial
  Synthetic deltaLNGFRco sequence in reverse orientation
tcacaggatgccccggttccaccgcttgaaggcgatataggccaccaggcccacgaccacagcggccaggatgctgcagtacacggggatcaggttgtcggtggtgcctcttgtcacgacgggctgagaactgcccatcactgtggtcacgacgccggccactgtagaggcgatcagatcctgctcaggaggggcctcaggttcctgggtgctaggggctgtgctatcgctgccctcagggggggtgcttctggtgatccatctgccagggatctcttcgcactcggcatcggcccatctggtacactcgcgcagctgccgctcggtatcttcgcacactgtgcatggcaggcaggggtccacgtgattggcctcgtcgctgtaggtgccgtcggggcactcttcacacacggtgttctgcttgtcttgacaactgaacaccaggccagatccggcctcgcacactctacaggcctcgcatctgccggttgtctcgtcctggtagtagccgtaggcgcatctgcacacggcatcgtcggcttccacgcaaggggcgctcatgctctgcaggcccacacactcggtacaaggcttgcaaggctcggtggcggacaccacgtcgctaaaggtcacgctgtccaggcagggctcgcacacggtctgattagcgccgcaaggctgggccacgccttctcccagattgcaggccttgcagcactcgccgctgtgtgtgtacaggccggtaggacaggcctctttggctccgcccagtgacacgcccagcagcaggagaaggaggagtctagggccatccat

SEQ ID NO: 9 - DNA, 592 bp, Artificial
  Synthetic WPRE sequence, LPREm6 [Sequence derived from WPRE J02442.1 region 1093-1684 with point mutations as described in Schambach et al Gene Therapy 2006]
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 10 - DNA, 250 bp, Artificial
  Synthetic polyadenylation signal in reverse orientation
cagatctgatcataatcagccataccacatttgtagaggttttacttgctttaaaaaacctcccacacctccccctgaacctgaaacataaaatgaatgcaattgttgttgttaacttgtttattgcagcttataatggttacaaataaggcaatagcatcacaaatttcacaaataaggcatttttttcactgcattctagttttggtttgtccaaactcatcaatgtatcttatcatgtctggatctc

SEQ ID NO: 11 - DNA, 9893 bp, Artificial
  Synthetic Whole sequence including the 5′and 3′ LTR sequences
ccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgattagaccagaaatagttcgtttaaaccagatctgatcataatcagccataccacatttgtagaggttttacttgctttaaaaaacctcccacacctccccctgaacctgaaacataaaatgaatgcaattgttgttgttaacttgtttattgcagcttataatggttacaaataaggcaatagcatcacaaatttcacaaataaggcatttttttcactgcattctagttttggtttgtccaaactcatcaatgtatcttatcatgtctggatctcaaatccctcggaagctgcgcctgtcatcaattcctgcagcccggtgcatgactaatcagttagcctcccccatctccctcgactcctgcaggctatcacaggatgccccggttccaccgcttgaaggcgatataggccaccaggcccacgaccacagcggccaggatgctgcagtacacggggatcaggttgtcggtggtgcctcttgtcacgacgggctgagaactgcccatcactgtggtcacgacgccggccactgtagaggcgatcagatcctgctcaggaggggcctcaggttcctgggtgctaggggctgtgctatcgctgccctcagggggggtgcttctggtgatccatctgccagggatctcttcgcactcggcatcggcccatctggtacactcgcgcagctgccgctcggtatcttcgcacactgtgcatggcaggcaggggtccacgtgattggcctcgtcgctgtaggtgccgtcggggcactcttcacacacggtgttctgcttgtcttgacaactgaacaccaggccagatccggcctcgcacactctacaggcctcgcatctgccggttgtctcgtcctggtagtagccgtaggcgcatctgcacacggcatcgtcggcttccacgcaaggggcgctcatgctctgcaggcccacacactcggtacaaggcttgcaaggctcggtggcggacaccacgtcgctaaaggtcacgctgtccaggcagggctcgcacacggtctgattagcgccgcaaggctgggccacgccttctcccagattgcaggccttgcagcactcgccgctgtgtgtgtacaggccggtaggacaggcctctttggctccgcccagtgacacgcccagcagcaggagaaggaggagtctagggccatccatggtggcacgcgtcgcgtcacgacacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagccttaattaaacgcctaccctcgagtagcttgatatgctagcccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccgggatccgccaccatgcccaaccccagacccggaaagcctagcgccccttctctggccctgggaccttctcctggcgcctccccatcttggagagccgcccctaaagccagcgatctgctgggagctagaggccctggcggcacattccagggcagagatctgagaggcggagcccacgcctctagcagcagcctgaatcccatgccccctagccagctgcagctgcctacactgcctctcgtgatggtggcccctagcggagctagactgggccctctgcctcatctgcaggccctgctgcaggacagaccccacttcatgcaccagctgagcaccgtggatgcccacgccagaacacctgtgctgcaggtgcaccccctggaaagccctgccatgatcagcctgacccctccaaccacagccaccggcgtgttcagcctgaaggccagacctggactgccccctggcatcaatgtggccagcctggaatgggtgtcccgcgaacctgccctgctgtgcaccttccccaatcccagcgcccccagaaaggacagcacactgtctgccgtgccccagagcagctatcccctgctggctaacggcgtgtgcaagtggcctggctgcgagaaggtgttcgaggaacccgaggacttcctgaagcactgccaggccgaccatctgctggacgagaaaggcagagcccagtgtctgctgcagcgcgagatggtgcagagcctggaacagcagctggtgctggaaaaagaaaagctgagcgccatgcaggcccacctggccggaaaaatggccctgacaaaggccagcagcgtggccagctctgacaagggcagctgctgcattgtggccgctggctctcagggacctgtggtgcctgcttggagcggacctagagaggcccccgatagcctgtttgccgtgcggagacacctgtggggcagccacggcaactctaccttccccgagttcctgcacaacatggactacttcaagttccacaacatgaggccccccttcacctacgccaccctgatcagatgggccattctggaagcccccgagaagcagcggaccctgaacgagatctaccactggtttacccggatgttcgccttcttccggaaccaccccgccacctggaagaacgccatccggcacaatctgagcctgcacaagtgcttcgtgcgggtggaaagcgagaagggcgccgtgtggacagtggacgagctggaatttcggaagaagcggtcccagaggcccagccggtgtagcaatcctacccctggcccttgataggcatgcatatggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttgg

(19) United States
(12) Patent Application Publication
(10) Pub. No.: US 2022/0136006 A1
(43) Pub. Date: May 5, 2022

(54) RECOMBINANT VECTORS SUITABLE FOR THE TREATMENT OF IPEX SYNDROME

(71) Applicants: INSERM (INSTITUT NATIONAL DE LA SANTÉ ET DE LA RECHERCHE MÉDICALE), Paris (FR); ASSISTANCE PUBLIQUE-HÔPITAUX DE PARIS (APHP), Paris (FR); FONDATION IMAGINE, Paris (FR); UNIVERSITÉ DE PARIS, Paris (FR); UNIVERSITÉ D'EVRY-VAL-D'ESSONE, Evry (FR); MEDIZINISCHE HOCHSCHULE HANNOVER (MHH), Hannover (DE)

(72) Inventors: Isabelle ANDRE, Paris (FR); Emmanuelle SIX, Paris (FR); Florence BELLIER, Paris (FR); Marianne DELVILLE, Paris (FR); Mariana CAVAZZANA, Paris (FR); Mario AMENDOLA, Paris (FR); Axel SCHAMBACH, Hannover (DE)

(21) Appl. No.: 17/428,401

(22) PCT Filed: Feb. 4, 2020
(86) PCT No.: PCT/EP2020/052731

§ 371 Date: Aug. 4, 2021

(30) Foreign Application Priority Data
Feb. 5, 2019 (EP) 19305133.1

(51) Int. Cl. C12N 15/86; A61K 35/17

(52) U.S. Cl. CPC C12N 15/86; A61K 35/17; C12N 2740/16043; C12N 2830/48; C12N 2830/205

(57) ABSTRACT

IPEX (Immune dysregulation Polyendocinopathy X linked) syndrome is a primary immunodeficience caused by mutations in the gene encoding the transcription factor forkhead box P3 (FOXP3), which leads to the loss of function of thymus-derived CD4+CD25+ regulatory T (tTreg) cells. Preclinical and clinical studies suggest that T cell gene therapy approaches designed to selectively restore the repertoire of Treg cells by transfer of wild type FOXP3 gene is a promising potential cure for IPEX. However, there is still a need for a vector that can be used efficiently for the preparation of said Treg cells. The inventors thus compared 6 different lentiviral constructs according to 4 criteria (vector titers, level of transduction of human CD4+ T cells, level of expression of FOXP3 and ΔLNGFR genes, degree of correlation between both expression) and selected one construct comprising a bidirectional EFS-PGK promoter that showed remarkable efficiency.

### FIELD OF THE INVENTION

The present invention relates to recombinant vectors suitable for the treatment of IPEX syndrome.

### BACKGROUND OF THE INVENTION

IPEX (Immune dysregulation Polyendocinopathy X linked) syndrome is a primary immunodeficience caused by mutations in the gene encoding the transcription factor forkhead box P3 (FOXP3) (Wildin et al., 2001) (Bennett et al., 2001), which leads to the loss of function of thymus-derived CD4+CD25+ regulatory T (tTreg) cells (Yagi et al., 2004) (Fontenot et al., 2003) (Hori et al., 2003) (Khattri et al., 2003) (a small subset of circulating CD4+T lymphocytes dedicated to controlling immune responses to self and foreign antigens). In IPEX patients, the absence of a functional Treg cell compartment leads to the development of multiple autoimmune manifestations (including severe enteropathy, type 1 diabetes and eczema) in the first months or years of life (Barzaghi et al., 2012). IPEX syndrome is often fatal early in infancy, and so a prompt diagnosis is essential for starting treatment as soon as possible (before tissue damage spreads to multiple organs).

The current treatments for IPEX syndrome include supportive therapy, immunosuppressive therapy, hormone replacement therapy and HSCT. Unfortunately, these immunosuppressants are usually only partially effective and the dose is often limited by infectious complications and toxicity. Currently, the only cure for IPEX syndrome is allogeneic HSCT. The absence of an HLA-compatible donor for all patients and their poor clinical condition particularly expose them to a risk of mortality. For all these reasons, effective alternative therapeutic approaches are urgently needed.

Based on the outcome of HSCT in this setting, we learned that partial donor chimerism is sufficient for complete remission—provided that full engraftment is achieved in the Treg compartment. In turn, this suggests that a few Tregs could be enough to control autoimmunity in IPEX syndrome (Horino et al., 2014) (Seidel et al., 2009) (Kasow et al., 2011). Moreover, various studies in the mouse (including the scurfy mouse model) have demonstrated the efficacy of the adoptive transfer of healthy Tregs in curing autoimmune diseases (Fontenot et al., 2003) (Mottet et al., 2003) (Tang et al., 2004). The in vivo suppressive capacity of human Tregs obtained after ex vivo expansion has also been demonstrated using humanized mouse model (Wieckiewicz et al., 2010). These various preclinical studies have paved the way for the first clinical trial of the adoptive transfer of ex vivo-expanded Treg cells in two patients with GVHD (Trzonkowski et al., 2009).

Gene therapy of T cells has been successfully developed for TCR or chimeric antigen receptor gene therapy and effectively targets cancer (Bonini et al., 2011) (Kalos and June, 2013) (Provasi et al., 2012). Previous experience of cell therapy with gene-modified T-cells in ADA-SCID (Aiuti et al., 2002) (Blaese et al., 1995) (Muul et al., 2003) indicates that gene-corrected functional T cells persist for more than 15 years after infusion. Furthermore, it has been demonstrated that LV-mediated FOXP3 expression in human CD4 T cells, including from IPEX patients enables the generation of regulatory T cells, which exhibited immunossuppressive activity both in vitro and in vivo in a xenogenic model of GVHD (Aarts-Riemens et al., 2008) (Allan et al., 2008)(Passerini et al., 2013).

Altogether the results of these preclinical and clinical studies suggest that T cell gene therapy approaches designed to selectively restore the repertoire of Treg cells by transfer of wild type FOXP3 gene is a promising potential cure for IPEX (Aiuti et al., 2012).

However, several prerequisites are absolutely required before any clinical application. First, FOXP3 controls partly the transcriptional signature—and therefore the suppressive function- of Tregs. It has to be expressed at sufficient level to ensure this function and stably to avoid any conversion from Treg to T effector cells and loss of suppressive ability. Secondly, the in vitro generated Tregs must be sorted before their infusion to the patients to avoid any side-effect of non-corrected contaminant effector T cells. As FOXP3 protein is located in the nucleus, it cannot be used to sort FOXP3+ expressing cells. Therefore, it has to be co-expressed with a surface marker. A truncated form of the p75 low-affinity nerve growth factor receptor (ΔLNGFR) with most of the intracytoplasmic tail deleted (from residue 248) has been used as a surface marker in T-cell targeted gene therapy approaches without any side effect (Bonini et al, 1997). Furthermore, the expression of surface ΔLNGFR allows the sorting of transduced cells in clinically applicable conditions. However, to be applicable, the correlation between ΔLNGFR and FOXP3 expression has to be perfect and ΔLNGFR expression should be sufficient to allow the sorting of ΔLNGFR+ cells. The different localization of the two proteins, FOXP3 in the nucleus and ΔLNGFR at the membrane might hamper a strict correlation between both expressions. Thirdly, another obstacle to gene therapy is the efficacy of lentiviral vector production (measured by titrating the vector), which is highly variable and depends on the vector construct and the transgenes. From our knowledge and long-lasting experience, it is impossible to predict the titer. Accordingly, there is still a need for a vector that addresses all these obstacles.

### SUMMARY OF THE INVENTION

The present invention relates to recombinant vectors suitable for the treatment of IPEX syndrome. In particular, the present invention is defined by the claims.

### DETAILED DESCRIPTION OF THE INVENTION

As shown in the EXAMPLE, the inventors compared 6 different lentiviral constructs according to 4 criteria (vector titers, level of transduction of human CD4+ T cells, level of expression of FOXP3 and ΔLNGFR genes, degree of correlation between both expression) and selected one construct comprising a bidirectional EFS-PGK promoter that showed remarkable efficiency.

Accordingly, the first object of the present invention relates to a recombinant nucleic acid molecule comprising a bidirectional EFS-PGK promoter operably linked to a first transgene in one direction and to a second transgene in the opposite direction wherein the first transgene that is under the control of the EFS portion of the bidirectional promoter encodes for a protein that is not constitutively expressed by a T cell and the second transgene that is under the control of the PGK portion of the bidirectional promoter encodes for a transcription factor.

As used herein, the term “nucleic acid molecule” has its general meaning in the art and refers to a DNA molecule.

As used herein, the terms “promoter” has its general meaning in the art and refers to a segment of a nucleic acid sequence, typically but not limited to DNA that controls the transcription of the nucleic acid sequence to which it is operatively linked. The promoter region includes specific sequences that are sufficient for RNA polymerase recognition, binding and transcription initiation. In addition, the promoter region can optionally include sequences which modulate this recognition, binding and transcription initiation activity of RNA polymerase. The skilled person will be aware that promoters are built from stretches of nucleic acid sequences and often comprise elements or functional units in those stretches of nucleic acid sequences, such as a transcription start site, a binding site for RNA polymerase, general transcription factor binding sites, such as a TATA box, specific transcription factor binding sites, and the like. Further regulatory sequences may be present as well, such as enhancers, and sometimes introns at the end of a promoter sequence.

As used herein, the term “EFS promoter” has its general meaning in the art and refers to the promoter of the gene encoding for an intron-less promoter derived from elongation factor-1 alpha promoter. An exemplary nucleic acid sequence for the EFS promoter is represented by SEQ ID NO:1.

>EFS promoter
SEQ ID NO: 1
ggctccggtgcccgtcagtgggcagagcgcacatcgcccacagtccccgag
aagttggggggaggggtcggcaattgaaccggtgcctagagaaggtggcgc
ggggtaaactgggaaagtgatgtcgtgtactggctccgcctttttcccgag
ggtgggggagaaccgtatataagtgcagtagtcgccgtgaacgttcttttt
cgcaacgggtttgccgccagaacacaggtgtcgtgacgc

As used herein, the term “PGK promoter” has its general meaning in the art and refers to the promoter of the gene encoding for phosphoglycerate kinase. An exemplary nucleic acid sequence for the PGK promoter is represented by SEQ ID NO:2.

SEQ ID ID NO: 2 >PGK promoter ccacqqqqttqqqqttqcqccttttccaaqqcaqccctqqqtttqcqcaqqqacqcqqctqctctqqqc gtggttccgggaaacgcagcggcgccgaccctgggtctcgcacattcttcacgtccgttcgcagcgtca cccggatcttcgccgctacccttgtgggccccccggcgacgcttcctgctccgcccctaagtcgggaag gttccttgcggttcgcggcgtgccggacgtgacaaacggaagccgcacgtctcactagtaccctcgcag acqqacaqcqccaqqqaqcaatqqcaqcqcqccqaccqcqatqqqctqtqqccaataqcqqctqctcaq cqqqqcqcqccgagaqcaqcqqccqqqaaqqqqcqqtqcqqqaqqcqqqqtqtqqqqcqqtaqtqtqqg ccctgttcctgcccgcgcggtgttccgcattctgcaagcctccggagcgcacgtcggcagtcggctccc tcgttgaccgaatcaccgacctctctcccc

As used herein, the term “bidirectional promoter” has its general meaning in the art and refers to a promoter which directs transcription of at least 2 transgenes in opposite orientations. Accordingly, a bidirectional promoter according to the present invention directs transcription of a first transgene which lies 5′ to 3′ in the same 5′ to 3′ direction as said promoter (“forward orientation”) and also directs transcription of another transgene which lies 5′ to 3′ in a direction opposite from the 5′ to 3′ direction of said promoter (“reverse orientation”). The bidirectional promoter of the present invention direct gene expression in a bidirectional fashion controlling expression for transgenes placed on both sides of the bidirectional promoter sequence. Thus, the recombinant nucleic acid molecule of the present invention comprises two transgenes, wherein the transcriptional direction (5′ to 3′) of the EFS and PGK portions of the EFS-PGK bidirectional promoter point away from each other (head to head configuration), wherein a first transgene is operably linked in one direction on the left side (i.e. in a reverse orientation), with expression controlled by the EFS portion of the bidirectional promoter, and a second transgene is operably linked in the opposite direction on the right side (i.e. in a forward orientation), with expression controlled by the PGK portion of the bidirectional promoter.

According to the present invention, the bidirectional promoter of the present invention comprises a first portion that derives from the EFS promoter and a second portion derives from the PGK promoter.

In some embodiments, the first portion that derives from the EFS promoter comprises a nucleic sequence having at least 80% of identity with the nucleic acid sequence as set forth in SEQ ID NO:3 (i.e. the nucleic acid sequence as set forth in SEQ ID NO:1 that is reverse orientated).

>EFS promoter in reverse orientation
SEQ ID NO: 3
gcgtcacgacacctgtgttctggcggcaaacccgttgcgaaaaagaacgt
tcacggcgactactgcacttatatacggttctcccccaccctcgggaaaa
aggcggagccagtacacgacatcactttcccagtttaccccgcgccacct
tctctaggcaccggttcaattgccgacccctccccccaacttctcgggga
ctgtgggcgatgtgcgctctgcccactgacgggcaccggagcc

In some embodiments, the second portion that derives from the PGK promoter comprises a nucleic sequence having at least 80% of identity with the nucleic acid sequence as set forth in SEQ ID NO:2.

In some embodiments, the first portion that derives from the EFS promoter and the second portion derives from the PGK promoter are separated by a spacer sequence. In some embodiments, the spacer sequence comprises a nucleic sequence having at least 80% of identity with the nucleic acid sequence as set forth in SEQ ID NO:4.

>linker
SEQ ID NO: 4

[symbol]

[symbol]

In some embodiments, the bidirectional promoter of the present invention comprises a nucleic acid sequence having at least 80% of identity with the sequence as set forth in SEQ ID NO:5.

>bidirectional promoter
SEQ ID NO: 5
gcgtcacgacacctgtgttctggcggcaaacccgttgcgaaaaagaacgttcacggcgactactgcact
tatatacggttctcccccaccctcgggaaaaaggcggagccagtacacgacatcactttcccagtttac
cccgcgccaccttctctaggcaccggttcaattgccgacccctccccccaacttctcggggactgtggg
cgatgtgcgctctgcccactgacgggcaccggagcc [symbol] [symbol]
ccacggggttggggttgcgccttttccaaggcagccctgggtttgcgcagggacgcggctgc
tctgggcgtggttccgggaaacgcagcggcgccgaccctgggtctcgcacattcttcacgtccgttcgc
agcgtcacccggatcttcgccgctacccttgtgggccccccggcgacgcttcctgctccgcccctaagt
cgggaaggttccttgcggttcgcggcgtgccggacgtgacaaacggaagccgcacgtctcactagtacc
ctcgcagacggacagcgccagggagcaatggcagcgcgccgaccgcgatgggctgtggccaatagcggc
tgctcagcggggcgcgccgagagcagcggccgggaaggggcggtgcgggaggcggggtgtggggcggta
gtgtgggccctgttcctgcccgcgcggtgttccgcattctgcaagcctccggagcgcacgtcggcagtc
ggctccctcgttgaccgaatcaccgacctctctcccc

According to the invention a first nucleic acid sequence having at least 80% of identity with a second nucleic acid sequence means that the first sequence has 80, 81, 82, 83, 84, 85, 86, 87, 88, 89, 90; 91; 92; 93; 94; 95; 96; 97; 98; 99 or 100% of identity with the second nucleic acid sequence.

As used herein, the term “sequence identity,” as used herein, has the standard meaning in the art. As is known in the art, a number of different programs can be used to identify whether a nucleic acid sequence has sequence identity or similarity to another nucleic acid sequence. Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12:387 (1984), preferably using the default settings, or by inspection. An example of a useful algorithm is the BLAST algorithm, described in Altschul et al., J. Mol. Biol. 215:403 (1990) and Karlin et al., Proc. Natl. Acad. Sci. USA 90:5873 (1993). A particularly useful BLAST program is the WU-BLAST-2 program which was obtained from Altschul et al., Meth. Enzymol., 266:460 (1996); blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

As used herein, the term “transgene” refers to any nucleic acid that shall be expressed in a mammal cell, in particular a T cell.

In some embodiments, the sequence of the transgenes is codon-optimized. As used herein, the term “codon-optimized” refers to nucleic sequence that has been optimized to increase expression by substituting one or more codons normally present in a coding sequence (for example, in a wildtype sequence, including, e.g., a coding sequence for LNGFR or FoxP3) with a codon for the same (synonymous) amino acid. In this manner, the protein encoded by the gene is identical, but the underlying nucleobase sequence of the gene or corresponding mRNA is different. In some embodiments, the optimization substitutes one or more rare codons (that is, codons for tRNA that occur relatively infrequently in cells from a particular species) with synonymous codons that occur more frequently to improve the efficiency of translation. For example, in human codon-optimization one or more codons in a coding sequence are replaced by codons that occur more frequently in human cells for the same amino acid. Codon optimization can also increase gene expression through other mechanisms that can improve efficiency of transcription and/or translation. Strategies include, without limitation, increasing total GC content (that is, the percent of guanines and cytosines in the entire coding sequence), decreasing CpG content (that is, the number of CG or GC dinucleotides in the coding sequence), removing cryptic splice donor or acceptor sites, and/or adding or removing ribosomal entry sites, such as Kozak sequences. Desirably, a codon-optimized gene exhibits improved protein expression, for example, the protein encoded thereby is expressed at a detectably greater level in a cell compared with the level of expression of the protein provided by the wildtype gene in an otherwise similar cell.

According to the present invention, the first transgene that is under the control of the EFS portion of the bidirectional promoter thus encodes for a protein that is not constitutively expressed by a T cell. Typically, the expression of said protein will be suitable for the cell sorting of the transformed cell with the recombinant nucleic acid molecule of the present invention ad described herein after. Typically, said protein is a cell surface marker so that use of binding partners specific for this protein can be used for cell sorting. In some embodiments, the protein is a receptor that will be expressed at the surface of the T cell. In some embodiments, the protein derives from the LNGFR. As used herein, the term “LNGFR” has its general meaning in the art and refers to the low-affinity nerve growth factor receptor. It is a member of the Tumor Necrosis Factor receptor (TNFR) superfamily, and thus anonymously called TNFRSF16. ΔLNGFR consists of 427-amino-acid in overall length, and possesses an extracellular region with four 40 amino acid repeats with 6 cysteins at conserved positions followed by a serine/threonine-rich region, a single transmembrane domain, and a 155 amino acid cytoplasmic domain. LNGFR is expressed in a wide variety of tissues, such as brain, peripheral neurons, Schwann cells, liver, esophagus and oral epithelium and the mesenchyme. However, ΔLNGFR is not expressed in T cells. In some embodiments, the proteins consists of the LNGFR truncated of its intracytoplasmic part. This protein is named “ΔLNGFR”. In some embodiments, the first transgene comprises a nucleic acid sequence having at least 80% identity with the nucleic acid sequence as set forth in SEQ ID NO:6.

>deltaLNGERco sequence
SEQ ID NO: 6
atggatggccctagactcctccttctcctgctgctgggcgtgtcactgg
gcggagccaaagaggcctgtcctaccggcctgtacacacacagcggcga
gtgctgcaaggcctgcaatctgggagaaggcgtggcccagccttgcggc
gctaatcagaccgtgtgcgagccctgcctggacagcgtgacctttagcg
acgtggtgtccgccaccgagccttgcaagccttgtaccgagtgtgtggg
cctgcagagcatgagcgccccttgcgtggaagccgacgatgccgtgtgc
agatgcgcctacggctactaccaggacgagacaaccggcagatgcgagg
cctgtagagtgtgcgaggccggatctggcctggtgttcagttgtcaaga
caagcagaacaccgtgtgtgaagagtgccccgacggcacctacagcgac
gaggccaatcacgtggacccctgcctgccatgcacagtgtgcgaagata
ccgagcggcagctgcgcgagtgtaccagatgggccgatgccgagtgcga
agagatccctggcagatggatcaccagaagcaccccccctgagggcagc
gatagcacagcccctagcacccaggaacctgaggcccctcctgagcagg
atctgatcgcctctacagtggccggcgtcgtgaccacagtgatgggcag
ttctcagcccgtcgtgacaagaggcaccaccgacaacctgatccccgtg
tactgcagcatcctggccgctgtggtcgtgggcctggtggcctatatcg
ccttcaagcggtggaaccggggcatcctgtga

In some embodiments, the second transgene that is under the control of the PGK portion of the bidirectional promoter encodes for a transcription factor. In some embodiments, the transcription factor is FoxP3. As used herein, the term FoxP3 has its general meaning in the art and refers to a transcription factor belonging to the forkhead/winged-helix family of transcriptional regulators. FOXP3 appears to function as a master regulator (transcription factor) in the development and function of regulatory T cells. FoxP3 confers T cells with regulatory function and increases the expression of CTLA-4 and CD25, but decreases IL-2 production by acting as a transcriptional repressor. FoxP3 binds to and suppresses nuclear factor of activated T cells (NFAT) and nuclear factor-kappaB (NFKB) (Bettelli, E. M. et al, 2005, Proc Natl Acad Sci USA 102:5138). In some embodiments, the second transgene comprises a nucleic acid sequence having at least 80% identity with the nucleic acid sequence as set forth in SEQ ID NO:7.

>hFoxp3co sequence
SEQ ID NO: 7
atgcccaaccccagacccggaaagcctagcgccccttctctggccctgg
gaccttctcctggcgcctccccatcttggagagccgcccctaaagccag
cgatctgctgggagctagaggccctggcggcacattccagggcagagat
ctgagaggcggagcccacgcctctagcagcagcctgaatcccatgcccc
ctagccagctgcagctgcctacactgcctctcgtgatggtggcccctag
cggagctagactgggccctctgcctcatctgcaggccctgctgcaggac
agaccccacttcatgcaccagctgagcaccgtggatgcccacgccagaa
cacctgtgctgcaggtgcaccccctggaaagccctgccatgatcagcct
gacccctccaaccacagccaccggcgtgttcagcctgaaggccagacct
ggactgccccctggcatcaatgtggccagcctggaatgggtgtcccgcg
aacctgccctgctgtgcaccttccccaatcccagcgcccccagaaagga
cagcacactgtctgccgtgccccagagcagctatcccctgctggctaac
ggcgtgtgcaagtggcctggctgcgagaaggtgttcgaggaacccgagg
acttcctgaagcactgccaggccgaccatctgctggacgagaaaggcag
agcccagtgtctgctgcagcgcgagatggtgcagagcctggaacagcag
ctggtgctggaaaaagaaaagctgagcgccatgcaggcccacctggccg
gaaaaatggccctgacaaaggccagcagcgtggccagctctgacaaggg
cagctgctgcattgtggccgctggctctcagggacctgtggtgcctgct
tggagcggacctagagaggcccccgatagcctgtttgccgtgcggagac
acctgtggggcagccacggcaactctaccttccccgagttcctgcacaa
catggactacttcaagttccacaacatgaggccccccttcacctacgcc
accctgatcagatgggccattctggaagcccccgagaagcagcggaccc
tgaacgagatctaccactggtttacccggatgttcgccttcttccggaa
ccaccccgccacctggaagaacgccatccggcacaatctgagcctgcac
aagtgcttcgtgcgggtggaaagcgagaagggcgccgtgtggacagtgg
acgagctggaatttcggaagaagcggtcccagaggcccagccggtgtag
caatcctacccctggcccttga

In some embodiments, the nucleic acid molecule of the present invention comprises:
i) a first nucleic acid sequence having at least 80% of identity with the nucleic acid sequence as set forth in SEQ ID: 8 (which corresponds to the nucleic acid sequence encoding for ΔLNGFR (i.e. SEQ ID NO:6) a in reverse orientation),
ii) a second nucleic acid sequence having at least 80% of identity with the nucleic acid sequence acid sequence as set forth in SEQ ID NO:5 (which corresponds to the bidirectional promoter) and
iii) a third nucleic acid sequence having at least 80% of identity with the nucleic acid sequence as set forth in SEQ ID NO:6 (which corresponds to the nucleic acid sequence encoding for FoxP3).

>deltaLNGFRco sequence in reverse orientation
SEQ ID NO: 8
tcacaggatgccccggttccaccgcttgaaggcgatataggccaccagg
cccacgaccacagcggccaggatgctgcagtacacggggatcaggttgt
cggtggtgcctcttgtcacgacgggctgagaactgcccatcactgtggt
cacgacgccggccactgtagaggcgatcagatcctgctcaggaggggcc
tcaggttcctgggtgctaggggctgtgctatcgctgccctcaggggggg
tgcttctggtgatccatctgccagggatctcttcgcactcggcatcggc
ccatctggtacactcgcgcagctgccgctcggtatcttcgcacactgtg
catggcaggcaggggtccacgtgattggcctcgtcgctgtaggtgccgt
cggggcactcttcacacacggtgttctgcttgtcttgacaactgaacac
caggccagatccggcctcgcacactctacaggcctcgcatctgccggtt
gtctcgtcctggtagtagccgtaggcgcatctgcacacggcatcgtcgg
cttccacgcaaggggcgctcatgctctgcaggcccacacactcggtaca
aggcttgcaaggctcggtggcggacaccacgtcgctaaaggtcacgctg
tccaggcagggctcgcacacggtctgattagcgccgcaaggctgggcca
cgccttctcccagattgcaggccttgcagcactcgccgctgtgtgtgta
caggccggtaggacaggcctctttggctccgcccagtgacacgcccagc
agcaggagaaggaggagtctagggccatccat

As used herein, the terms “operably linked”, or “operatively linked” are used interchangeably herein, and refer to the functional relationship of the nucleic acid sequences with regulatory sequences of nucleotides, such as promoters, enhancers, transcriptional and translational stop sites, and other signal sequences and indicates that two or more DNA segments are joined together such that they function in concert for their intended purposes. For example, operative linkage of nucleic acid sequences, typically DNA, to a regulatory sequence or promoter region refers to the physical and functional relationship between the DNA and the regulatory sequence or promoter such that the transcription of such DNA is initiated from the regulatory sequence or promoter, by an RNA polymerase that specifically recognizes, binds and transcribes the DNA. In order to optimize expression and/or in vitro transcription, it may be necessary to modify the regulatory sequence for the expression of the nucleic acid or DNA in the cell type for which it is expressed. The desirability of, or need of, such modification may be empirically determined.

Further regulatory sequences may also be added to the recombinant nucleic acid molecule of the present invention. As used herein, the term “regulatory sequence” is used interchangeably with “regulatory element” herein and refers to a segment of nucleic acid, typically but not limited to DNA, that modulate the transcription of the nucleic acid sequence to which it is operatively linked, and thus acts as a transcriptional modulator. A regulatory sequence often comprises nucleic acid sequences that are transcription binding domains that are recognized by the nucleic acid-binding domains of transcriptional proteins and/or transcription factors, enhancers or repressors etc. In some embodiments, the nucleic acid molecule of the present invention comprises a Woodchuck Hepatitis Virus (WHP) Posttranscriptional Regulatory Element (WPRE) sequence that is a DNA sequence that, when transcribed creates a tertiary structure enhancing expression, by stabilization of the messenger RNA. Typically, the WPRE sequence is inserted downstream to the second transgene (e.g. FoxP3). In some embodiments, the recombinant acid molecule of the present invention comprises a WPRE sequence devoid of X protein open reading frames (ORFs), that allows to remove oncogenic side effect without significant loss of RNA enhancement activity (Schambach, A. et al. Woodchuck hepatitis virus post-transcriptional regulatory element deleted from X protein and promoter sequences enhances retroviral vector titer and expression. Gene Ther. 13,641-645 (2006)). In some embodiments, the WPRE sequence comprises nucleic acid sequence having at least 80% of identity with the nucleic acid sequence as set forth in SEQ ID NO: 9.

>WPRE sequence, LPREm6 [Sequence derived from
WPRE J02442.1 region 1093-1684 with point muta-
tions as described in Schambach et al Gene
Therapy 2006]
SEQ ID NO: 9
AATCAACCTCTGGATTACAAAATTTGTGAAAGATTGACTGGTATTCTTA
ACTATGTTGCTCCTTTTACGCTATGTGGATACGCTGCTTTAATGCCTTT
GTATCATGCTATTGCTTCCCGTATGGCTTTCATTTTCTCCTCCTTGTAT
AAATCCTGGTTGCTGTCTCTTTATGAGGAGTTGTGGCCCGTTGTCAGGC
AACGTGGCGTGGTGTGCACTGTGTTTGCTGACGCAACCCCCACTGGTTG
GGGCATTGCCACCACCTGTCAGCTCCTTTCCGGGACTTTCGCTTTCCCC
CTCCCTATTGCCACGGCGGAACTCATCGCCGCCTGCCTTGCCCGCTGCT
GGACAGGGGCTCGGCTGTTGGGCACTGACAATTCCGTGGTGTTGTCGGG
GAAATCATCGTCCTTTCCTTGGCTGCTCGCCTGTGTTGCCACCTGGATT
CTGCGCGGGACGTCCTTCTGCTACGTCCCTTCGGCCCTCAATCCAGCGG
ACCTTCCTTCCCGCGGCCTGCTGCCGGCTCTGCGGCCTCTTCCGCGTCT
TCGCCTTCGCCCTCAGACGAGTCGGATCTCCCTTTGGGCCGCCTCCCCG
CCTG

In some embodiments, the recombinant nucleic acid molecule of the present invention comprises a polyadenylation signal sequence inserted downstream to the first transgene (e.g. ΔLNGFR). As used herein, the term “polyadenylation signal sequence” has its general meaning in the art and refers to a nucleic acid sequence that mediates the attachment of a polyadenine stretch to the 3′ terminus of the mRNA. Suitable polyadenylation signals include the SV40 early polyadenylation signal, the SV40 late polyadenylation signal, the HSV thymidine kinase polyadenylation signal, the protamine gene polyadenylation signal, the adenovirus 5 EIb polyadenylation signal, the bovine growth hormone polyadenylation signal, the human variant growth hormone polyadenylation signal and the like. In some embodiments, the polyadenylation sequence comprises nucleic acid sequence having at least 80% of identity with the nucleic acid sequence as set forth in SEQ ID NO: 10.

>polyadenylation signal in reverse orientation
SEQ ID NO: 10
cagatctgatcataatcagccataccacatttgtagaggttttacttgc
tttaaaaaacctcccacacctccccctgaacctgaaacataaaatgaat
gcaattgttgttgttaacttgtttattgcagcttataatggttacaaat
aaggcaatagcatcacaaatttcacaaataaggcatttttttcactgca
ttctagttttggtttgtccaaactcatcaatgtatcttatcatgtctgg
atctc

In some embodiments, the recombinant acid molecule of the present invention comprises a nucleic acid sequence having at least 80% of identity with the nucleic acid sequence as set forth in SEQ ID NO:11.

>Whole sequence including the 5′ and 3′ LTR sequences
SEQ ID NO: 11
ccattgcatacgttgtatccatatcataatatgtacatttatattggctcatgtccaacattaccgcca
tgttgacattgattattgactagttattaatagtaatcaattacggggtcattagttcatagcccatat
atggagttccgcgttacataacttacggtaaatggcccgcctggctgaccgcccaacgacccccgccca
ttgacgtcaataatgacgtatgttcccatagtaacgccaatagggactttccattgacgtcaatgggtg
gagtatttacggtaaactgcccacttggcagtacatcaagtgtatcatatgccaagtacgccccctatt
gacgtcaatgacggtaaatggcccgcctggcattatgcccagtacatgaccttatgggactttcctact
tggcagtacatctacgtattagtcatcgctattaccatggtgatgcggttttggcagtacatcaatggg
cgtggatagcggtttgactcacggggatttccaagtctccaccccattgacgtcaatgggagtttgttt
tggcaccaaaatcaacgggactttccaaaatgtcgtaacaactccgccccattgacgcaaatgggcggt
aggcgtgtacggtgggaggtctatataagcagagctcgtttagtgaaccggggtctctctggttagacc
agatctgagcctgggagctctctggctaactagggaacccactgcttaagcctcaataaagcttgcctt
gagtgcttcaagtagtgtgtgcccgtctgttgtgtgactctggtaactagagatccctcagaccctttt
agtcagtgtggaaaatctctagcagtggcgcccgaacagggacttgaaagcgaaagggaaaccagagga
gctctctcgacgcaggactcggcttgctgaagcgcgcacggcaagaggcgaggggcggcgactggtgag
tacgccaaaaattttgactagcggaggctagaaggagagagatgggtgcgagagcgtcagtattaagcg
ggggagaattagatcgcgatgggaaaaaattcggttaaggccagggggaaagaaaaaatataaattaaa
acatatagtatgggcaagcagggagctagaacgattcgcagttaatcctggcctgttagaaacatcaga
aggctgtagacaaatactgggacagctacaaccatcccttcagacaggatcagaagaacttagatcatt
atataatacagtagcaaccctctattgtgtgcatcaaaggatagagataaaagacaccaaggaagcttt
agacaagatagaggaagagcaaaacaaaagtaagaccaccgcacagcaagcggccgctgatcttcagac
ctggaggaggagatatgagggacaattggagaagtgaattatataaatataaagtagtaaaaattgaac
cattaggagtagcacccaccaaggcaaagagaagagtggtgcagagagaaaaaagagcagtgggaatag
gagctttgttccttgggttcttgggagcagcaggaagcactatgggcgcagcctcaatgacgctgacgg
tacaggccagacaattattgtctggtatagtgcagcagcagaacaatttgctgagggctattgaggcgc
aacagcatctgttgcaactcacagtctggggcatcaagcagctccaggcaagaatcctggctgtggaaa
gatacctaaaggatcaacagctcctggggatttggggttgctctggaaaactcatttgcaccactgctg
tgccttggaatgctagttggagtaataaatctctggaacagatttggaatcacacgacctggatggagt
gggacagagaaattaacaattacacaagcttaatacactccttaattgaagaatcgcaaaaccagcaag
aaaagaatgaacaagaattattggaattagataaatgggcaagtttgtggaattggtttaacataacaa
attggctgtggtatataaaattattcataatgatagtaggaggcttggtaggtttaagaatagtttttg
ctgtactttctatagtgaatagagttaggcagggatattcaccattatcgtttcagacccacctcccaa
ccccgaggggacccgacaggcccgaaggaatagaagaagaaggtggagagagagacagagacagatcca
ttcgattagtgaacggatctcgacggtatcggttaacttttaaaagaaaaggggggattggggggtaca
gtgcaggggaaagaatagtagacataatagcaacagacatacaaactaaagaattacaaaaacaaatta
caaaaattcaaaattttatcgattagaccagaaatagttcgtttaaaccagatctgatcataatcagcc
ataccacatttgtagaggttttacttgctttaaaaaacctcccacacctccccctgaacctgaaacata
aaatgaatgcaattgttgttgttaacttgtttattgcagcttataatggttacaaataaggcaatagca
tcacaaatttcacaaataaggcatttttttcactgcattctagttttggtttgtccaaactcatcaatg
tatcttatcatgtctggatctcaaatccctcggaagctgcgcctgtcatcaattcctgcagcccggtgc
atgactaatcagttagcctcccccatctccctcgactcctgcaggctatcacaggatgccccggttcca
ccgcttgaaggcgatataggccaccaggcccacgaccacagcggccaggatgctgcagtacacggggat
caggttgtcggtggtgcctcttgtcacgacgggctgagaactgcccatcactgtggtcacgacgccggc
cactgtagaggcgatcagatcctgctcaggaggggcctcaggttcctgggtgctaggggctgtgctatc
gctgccctcagggggggtgcttctggtgatccatctgccagggatctcttcgcactcggcatcggccca
tctggtacactcgcgcagctgccgctcggtatcttcgcacactgtgcatggcaggcaggggtccacgtg
attggcctcgtcgctgtaggtgccgtcggggcactcttcacacacggtgttctgcttgtcttgacaact
gaacaccaggccagatccggcctcgcacactctacaggcctcgcatctgccggttgtctcgtcctggta
gtagccgtaggcgcatctgcacacggcatcgtcggcttccacgcaaggggcgctcatgctctgcaggcc
cacacactcggtacaaggcttgcaaggctcggtggcggacaccacgtcgctaaaggtcacgctgtccag
gcagggctcgcacacggtctgattagcgccgcaaggctgggccacgccttctcccagattgcaggcctt
gcagcactcgccgctgtgtgtgtacaggccggtaggacaggcctctttggctccgcccagtgacacgcc
cagcagcaggagaaggaggagtctagggccatccatggtggcacgcgtcgcgtcacgacacctgtgttc
tggcggcaaacccgttgcgaaaaagaacgttcacggcgactactgcacttatatacggttctcccccac
cctcgggaaaaaggcggagccagtacacgacatcactttcccagtttaccccgcgccaccttctctagg
caccggttcaattgccgacccctccccccaacttctcggggactgtgggcgatgtgcgctctgcccact
gacgggcaccggagccttaattaaacgcctaccctcgagtagcttgatatgctagcccacggggttggg
gttgcgccttttccaaggcagccctgggtttgcgcagggacgcggctgctctgggcgtggttccgggaa
acgcagcggcgccgaccctgggtctcgcacattcttcacgtccgttcgcagcgtcacccggatcttcgc
cgctacccttgtgggccccccggcgacgcttcctgctccgcccctaagtcgggaaggttccttgcggtt
cgcggcgtgccggacgtgacaaacggaagccgcacgtctcactagtaccctcgcagacggacagcgcca
gggagcaatggcagcgcgccgaccgcgatgggctgtggccaatagcggctgctcagcggggcgcgccga
gagcagcggccgggaaggggcggtgcgggaggcggggtgtggggcggtagtgtgggccctgttcctgcc
cgcgcggtgttccgcattctgcaagcctccggagcgcacgtcggcagtcggctccctcgttgaccgaat
caccgacctctctccccgggatccgccaccatgcccaaccccagacccggaaagcctagcgccccttct
ctggccctgggaccttctcctggcgcctccccatcttggagagccgcccctaaagccagcgatctgctg
ggagctagaggccctggcggcacattccagggcagagatctgagaggcggagcccacgcctctagcagc
agcctgaatcccatgccccctagccagctgcagctgcctacactgcctctcgtgatggtggcccctagc
ggagctagactgggccctctgcctcatctgcaggccctgctgcaggacagaccccacttcatgcaccag
ctgagcaccgtggatgcccacgccagaacacctgtgctgcaggtgcaccccctggaaagccctgccatg
atcagcctgacccctccaaccacagccaccggcgtgttcagcctgaaggccagacctggactgccccct
ggcatcaatgtggccagcctggaatgggtgtcccgcgaacctgccctgctgtgcaccttccccaatccc
agcgcccccagaaaggacagcacactgtctgccgtgccccagagcagctatcccctgctggctaacggc
gtgtgcaagtggcctggctgcgagaaggtgttcgaggaacccgaggacttcctgaagcactgccaggcc
gaccatctgctggacgagaaaggcagagcccagtgtctgctgcagcgcgagatggtgcagagcctggaa
cagcagctggtgctggaaaaagaaaagctgagcgccatgcaggcccacctggccggaaaaatggccctg
acaaaggccagcagcgtggccagctctgacaagggcagctgctgcattgtggccgctggctctcaggga
cctgtggtgcctgcttggagcggacctagagaggcccccgatagcctgtttgccgtgcggagacacctg
tggggcagccacggcaactctaccttccccgagttcctgcacaacatggactacttcaagttccacaac
atgaggccccccttcacctacgccaccctgatcagatgggccattctggaagcccccgagaagcagcgg
accctgaacgagatctaccactggtttacccggatgttcgccttcttccggaaccaccccgccacctgg
aagaacgccatccggcacaatctgagcctgcacaagtgcttcgtgcgggtggaaagcgagaagggcgcc
gtgtggacagtggacgagctggaatttcggaagaagcggtcccagaggcccagccggtgtagcaatcct
acccctggcccttgataggcatgcatatgGTCGACAATCAACCTCTGGATTACAAAATTTGTGAAAGAT
TGACTGGTATTCTTAACTATGTTGCTCCTTTTACGCTATGTGGATACGCTGCTTTAATGCCTTTGTATC
ATGCTATTGCTTCCCGTATGGCTTTCATTTTCTCCTCCTTGTATAAATCCTGGTTGCTGTCTCTTTATG
AGGAGTTGTGGCCCGTTGTCAGGCAACGTGGCGTGGTGTGCACTGTGTTTGCTGACGCAACCCCCACTG
GTTGGGGCATTGCCACCACCTGTCAGCTCCTTTCCGGGACTTTCGCTTTCCCCCTCCCTATTGCCACGG
CGGAACTCATCGCCGCCTGCCTTGCCCGCTGCTGGACAGGGGCTCGGCTGTTGGGCACTGACAATTCCG
TGGTGTTGTCGGGGAAATCATCGTCCTTTCCTTGGCTGCTCGCCTGTGTTGCCACCTGGATTCTGCGCG
GGACGTCCTTCTGCTACGTCCCTTCGGCCCTCAATCCAGCGGACCTTCCTTCCCGCGGCCTGCTGCCGG
CTCTGCGGCCTCTTCCGCGTCTTCGCCTTCGCCCTCAGACGAGTCGGATCTCCCTTTGGGCCGCCTCCC
CGCCTGGAATTCGAGCTCGGTACCtttaagaccaatgacttacaaggcagctgtagatcttagccactt
tttaaaagaaaaggggggactggaagggctaattcactcccaacgaagacaagatctgctttttgcttg
tactgggtctctctggttagaccagatctgagcctgggagctctctggctaactagggaacccactgct
taagcctcaataaagcttgccttgagtgcttcaagtagtgtgtgcccgtctgttgtgtgactctggtaa
ctagagatccctcagacccttttagtcagtgtggaaaatctctagcagtagtagttcatgtcatcttat
tattcagtatttataacttgcaaagaaatgaatatcagagagtgagaggaacttgtttattgcagctta
taatggttacaaataaagcaatagcatcacaaatttcacaaataaagcatttttttcactgcattctag
ttgtggtttgtccaaactcatcaatgtatcttatcatgtctggctctagctatcccgcccctaactccg
cccagttccgcccattctccgccccatggctgactaattttttttatttatgcagaggccgaggccgcc
tcggcctctgagctattccagaagtagtgaggaggcttttttggaggcctagggacgtacccaattcgc
cctatagtgagtcgtattacgcgcgctcactggccgtcgttttacaacgtcgtgactgggaaaaccctg
gcgttacccaacttaatcgccttgcagcacatccccctttcgccagctggcgtaatagcgaagaggccc
gcaccgatcgcccttcccaacagttgcgcagcctgaatggcgaatgggacgcgccctgtagcggcgcat
taagcgcggcgggtgtggtggttacgcctgaatggcgaatgggacgcgccctgtagcggcgcattaagc
gcggcgggtgtggtggttacgcgcagcgtgaccgctacacttgccagcgccctagcgcccgctcctttc
gctttcttcccttcctttctcgccacgttcgccggctttccccgtcaagctctaaatcgggggctccct
ttagggttccgatttagtgctttacggcacctcgaccccaaaaaacttgattagggtgatggttcacgt
agtgggccatcgccctgatagacggtttttcgccctttgacgttggagtccacgttctttaatagtgga
ctcttgttccaaactggaacaacactcaaccctatctcggtctattcttttgatttataagggattttg
ccgatttcggcctattggttaaaaaatgagctgatttaacaaaaatttaacgcgaattttaacaaaata
ttaacgcttacaatttaggtggcacttttcggggaaatgtgcgcggaacccctatttgtttatttttct
aaatacattcaaatatgtatccgctcatgagacaataaccctgataaatgcttcaataatattgaaaaa
ggaagagtatgagtattcaacatttccgtgtcgcccttattcccttttttgcggcattttgccttcctg
tttttgctcacccagaaacgctggtgaaagtaaaagatgctgaagatcagttgggtgcacgagtgggtt
acatcgaactggatctcaacagcggtaagatccttgagagttttcgccccgaagaacgttttccaatga
tgagcacttttaaagttctgctatgtggcgcggtattatcccgtattgacgccgggcaagagcaactcg
gtcgccgcatacactattctcagaatgacttggttgagtactcaccagtcacagaaaagcatcttacgg
atggcatgacagtaagagaattatgcagtgctgccataaccatgagtgataacactgcggccaacttac
ttctgacaacgatcggaggaccgaaggagctaaccgcttttttgcacaacatgggggatcatgtaactc
gccttgatcgttgggaaccggagctgaatgaagccataccaaacgacgagcgtgacaccacgatgcctg
tagcaatggcaacaacgttgcgcaaactattaactggcgaactacttactctagcttcccggcaacaat
taatagactggatggaggcggataaagttgcaggaccacttctgcgctcggcccttccggctggctggt
ttattgctgataaatctggagccggtgagcgtgggtctcgcggtatcattgcagcactggggccagatg
gtaagccctcccgtatcgtagttatctacacgacggggagtcaggcaactatggatgaacgaaatagac
agatcgctgagataggtgcctcactgattaagcattggtaactgtcagaccaagtttactcatatatac
tttagattgatttaaaacttcatttttaatttaaaaggatctaggtgaagatcctttttgataatctca
tgaccaaaatcccttaacgtgagttttcgttccactgagcgtcagaccccgtagaaaagatcaaaggat
cttcttgagatcctttttttctgcgcgtaatctgctgcttgcaaacaaaaaaaccaccgctaccagcgg
tggtttgtttgccggatcaagagctaccaactctttttccgaaggtaactggcttcagcagagcgcaga
taccaaatactgttcttctagtgtagccgtagttaggccaccacttcaagaactctgtagcaccgccta
catacctcgctctgctaatcctgttaccagtggctgctgccagtggcgataagtcgtgtcttaccgggt
tggactcaagacgatagttaccggataaggcgcagcggtcgggctgaacggggggttcgtgcacacagc
ccagcttggagcgaacgacctacaccgaactgagatacctacagcgtgagctatgagaaagcgccacgc
ttcccgaagggagaaaggcggacaggtatccggtaagcggcagggtcggaacaggagagcgcacgaggg
agcttccagggggaaacgcctggtatctttatagtcctgtcgggtttcgccacctctgacttgagcgtc
gatttttgtgatgctcgtcaggggggcggagcctatggaaaaacgccagcaacgcggcctttttacggt
tcctggccttttgctggccttttgctcacatgttctttcctgcgttatcccctgattctgtggataacc
gtattaccgcctttgagtgagctgataccgctcgccgcagccgaacgaccgagcgcagcgagtcagtga
gcgaggaagcggaagagcgcccaatacgcaaaccgcctctccccgcgcgttggccgattcattaatgca
gctggcacgacaggtttcccgactggaaagcgggcagtgagcgcaacgcaattaatgtgagttagctca
ctcattaggcaccccaggctttacactttatgcttccggctcgtatgttgtgtggaattgtgagcggat
aacaatttcacacaggaaacagctatgaccatgattacgccaagcgcgcaattaaccctcactaaaggg
aacaaaagctggagctgcaagcttgg

In some embodiments, the recombinant nucleic acid molecule of the present invention is inserted in a viral vector, and in particular in a retroviral vector.

As used herein, the term “viral vector” refer to a virion or virus particle that functions as a nucleic acid delivery vehicle and which comprises a vector genome packaged within the virion or virus particle.

As used herein, the term “retroviral vector” refers to a vector containing structural and functional genetic elements that are primarily derived from a retrovirus.

In some embodiments, the retroviral vector of the present invention derives from a retrovirus selected from the group consisting of alpharetroviruses (e.g., avian leukosis virus), betaretroviruses (e.g., mouse mammary tumor virus), gammaretroviruses (e.g., murine leukemia virus), deltaretroviruses (e.g., bovine leukemia virus), epsilonretroviruses (e.g., Walley dermal sarcoma virus), lentiviruses (e.g., HIV-1, HIV-2) and spumaviruses (e.g., human spumavirus).

In some embodiments, the retroviral vector of the present invention is a replication deficient retroviral virus particle, which can transfer a foreign imported RNA of a gene instead of the retroviral mRNA. In some embodiments, the retroviral vector of the present invention is a lentiviral vector.

As used herein, the term “lentiviral vector” refers to a vector containing structural and functional genetic elements that are primarily derived from a lentivirus. In some embodiments, the lentiviral vector of the present invention is selected from the group consisting of HIV-1, HIV-2, SIV, FIV, EIAV, BIV, VISNA and CAEV vectors. In some embodiments, the lentiviral vector is a HIV-1 vector.

The structure and composition of the vector genome used to prepare the retroviral vectors of the present invention are in accordance with those described in the art. Especially, minimum retroviral gene delivery vectors can be prepared from a vector genome, which only contains, apart from the recombinant nucleic acid molecule of the present invention, the sequences of the retroviral genome which are non-coding regions of said genome, necessary to provide recognition signals for DNA or RNA synthesis and processing. In some embodiment, the retroviral vector genome comprises all the elements necessary for the nucleic import and the correct expression of the polynucleotide of interest (i.e. the transgene). As examples of elements that can be inserted in the retroviral genome of the retroviral vector of the present invention are at least one (preferably two) long terminal repeats (LTR), such as a LTR5′ and a LTR3′, a psi sequence involved in the retroviral genome encapsidation, and optionally at least one DNA flap comprising a cPPT and a CTS domains. In some embodiments of the present invention, the LTR, preferably the LTR3′, is deleted for the promoter and the enhancer of U3 and is replaced by a minimal promoter allowing transcription during vector production while an internal promoter is added to allow expression of the transgene. In particular, the vector is a Self-INactivating (SIN) vector that contains a non-functional or modified 3′ Long Terminal Repeat (LTR) sequence. This sequence is copied to the 5′ end of the vector genome during integration, resulting in the inactivation of promoter activity by both LTRs. Hence, a vector genome may be a replacement vector in which all the viral coding sequences between the 2 long terminal repeats (LTRs) have been replaced by the recombinant nucleic acid molecule of the present invention.

In some embodiments, the retroviral vector genome is devoid of functional gag, pol and/or env retroviral genes. By “functional” it is meant a gene that is correctly transcribed, and/or correctly expressed. Thus, the retroviral vector genome of the present invention in this embodiment contains at least one of the gag, pol and env genes that is either not transcribed or incompletely transcribed; the expression “incompletely transcribed” refers to the alteration in the transcripts gag, gag-pro or gag-pro-pol, one of these or several of these being not transcribed. In some embodiments, the retroviral genome is devoid of gag, pol and/or env retroviral genes.

In some embodiments the retroviral vector genome is also devoid of the coding sequences for Vif-, Vpr-, Vpu- and Nef-accessory genes (for HIV-1 retroviral vectors), or of their complete or functional genes.

Typically, the retroviral vector of the present invention is non replicative i.e., the vector and retroviral vector genome are not able to form new particles budding from the infected host cell. This may be achieved by the absence in the retroviral genome of the gag, pol or env genes, as indicated in the above paragraph; this can also be achieved by deleting other viral coding sequence(s) and/or cis-acting genetic elements needed for particles formation.

The retroviral vectors of the present invention can be produced by any well-known method in the art including by transfection (s) transient (s), in stable cell lines and/or by means of helper virus. Use of stable cell lines may also be preferred for the production of the vectors (Greene, M. R. et al. Transduction of Human CD34+ Repopulating Cells with a Self-Inactivating Lentiviral Vector for SCID-X1 Produced at Clinical Scale by a Stable Cell Line. Hum. Gene Ther. Methods 23, 297-308 (2012).) For instance, the retroviral vector of the present invention is obtainable by a transcomplementation system (vector/packaging system) by transfecting in vitro a permissive cell (such as 293T cells) with a plasmid containing the retroviral vector genome of the present invention, and at least one other plasmid providing, in trans, the gag, pol and env sequences encoding the polypeptides GAG, POL and the envelope protein(s), or for a portion of these polypeptides sufficient to enable formation of retroviral particles. As an example, permissive cells are transfected with a) transcomplementation plasmid, lacking packaging signal psi and, the plasmid is optionally deleted of accessory genes vif, nef, vpu and/or vpr, b) a second plasmid (envelope expression plasmid or pseudo-typing env plasmid) comprising a gene encoding an envelope protein(s) and c) a plasmid vector comprising a recombinant genome retroviral, optionally deleted from the promoter region of the 3 ‘LTR or U3 enhancer sequence of the 3’ LTR, including, between the LTR sequences 5 ‘and 3’ retroviral, a psi encapsidation sequence, a nuclear export element (preferably RRE element of HIV or other retroviruses equivalent), comprising the nucleic acid molecule of the present invention and optionally a promoter and/or a nuclear import sequence (cPPT sequence eg CTS) of the RNA. Advantageously, the three plasmids used do not contain homologous sequence sufficient for recombination. Nucleic acids encoding gag, pol and env cDNA can be advantageously prepared according to conventional techniques, from viral gene sequences available in the prior art and databases. The trans-complementation plasmid provides a nucleic acid encoding the proteins retroviral gag and pol. These proteins are derived from a lentivirus, and most preferably, from HIV-1. The plasmid is devoid of encapsidation sequence, sequence coding for an envelope, accessory genes, and advantageously also lacks retroviral LTRs. Therefore, the sequences coding for gag and pol proteins are advantageously placed under the control of a heterologous promoter, eg cellular, viral, etc., which can be constitutive or regulated, weak or strong. It is preferably a plasmid containing a sequence transcomplémentant Δpsi-CMV-gag-pol-PolyA. This plasmid allows the expression of all the proteins necessary for the formation of empty virions, except the envelope glycoproteins. The plasmid transcomplementation may advantageously comprise the TAT and REV genes. Plasmid transcomplementation is advantageously devoid of vif, vpr, vpu and/or nef accessory genes. It is understood that the gag and pol genes and genes TAT and REV can also be carried by different plasmids, possibly separated. In this case, several plasmids are used transcomplementation, each encoding one or more of said proteins. The promoters used in the plasmid transcomplementation, the envelope plasmid and the plasmid vector respectively to promote the expression of gag and pol of the coat protein, the mRNA of the vector genome and the transgene are promoters identical or different, chosen advantageously from ubiquitous promoters or specific, for example, from viral promoters CMV, TK, RSV LTR promoter and the RNA polymerase III promoter such as U6 or H1 or promoters of helper viruses encoding env, gag and pol (i.e. adenoviral, baculoviral, herpes viruses). For the production of the retroviral vector of the present invention, the plasmids described above can be introduced into competent cells and viruses produced are harvested. The cells used may be any cell competent, particularly eukaryotic cells, in particular mammalian, eg human or animal. They can be somatic or embryonic stem or differentiated. Typically the cells include 293T cells, fibroblast cells, hepatocytes, muscle cells (skeletal, cardiac, smooth, blood vessel, etc.)., nerve cells (neurons, glial cells, astrocytes) of epithelial cells, renal, ocular etc. It may also include, insect, plant cells, yeast, or prokaryotic cells. It can also be cells transformed by the SV40 T antigen. The genes gag, pol and env encoded in plasmids or helper viruses can be introduced into cells by any method known in the art, suitable for cell type considered. Usually, the cells and the vector system are contacted in a suitable device (plate, dish, tube, pouch, etc. . . . ), for a period of time sufficient to allow the transfer of the vector system or the plasmid in the cells. Typically, the vector system or the plasmid is introduced into the cells by calcium phosphate precipitation, electroporation, transduction or by using one of transfection-facilitating compounds, such as lipids, polymers, liposomes and peptides, etc. The calcium phosphate precipitation is preferred. The cells are cultured in any suitable medium such as RPMI, DMEM, a specific medium to a culture in the absence of fetal calf serum, etc. Once transfected the retroviral vectors of the present invention may be purified from the supernatant of the cells. Purification of the retroviral vector to enhance the concentration can be accomplished by any suitable method, such as by density gradient purification (e.g., cesium chloride (CsCl)) or by chromatography techniques (e.g., column or batch chromatography). For example, the vector of the present invention can be subjected to two or three CsCl density gradient purification steps. The vector, is desirably purified from cells infected using a method that comprises lysing cells infected with adenovirus, applying the lysate to a chromatography resin, eluting the lentivirus from the chromatography resin, and collecting a fraction containing the retroviral vector of the present invention.

The vector of the present invention is particularly suitable for driving the targeted expression of the transgenes in T cells. According to the present invention, the expression of the transgenes is balanced from both sides of the bidirectional EFS-PGK promoter. As used herein, “balanced expression”, “balance of expression”, “expression balance”, or “balanced” as it refers to expression, mean that the expression from one side of the bidirectional promoter, as measured for example by different protein expression detection techniques such as Western Blot, FACS analysis, or other assays using luminescence or fluorescence, is comparable to the expression from the other side of the bidirectional promoter. Therefore, balanced expression of the 2 transgenes expressed by a bidirectional EFS-PGK promoter of the present invention is expected to generate comparable expression of both proteins.

In particular, the vector of the present invention is particularly suitable for obtaining a population of Treg cells that express LNGFR at their cell surface.

Thus a further object of the present invention relates to a method of producing a population of Treg cells, which comprises the step of transfecting or transducing a population of T cells in vitro or ex vivo with the vector of the present invention.

As used herein, the term “T cell” refers to a type of lymphocytes that play an important role in cell-mediated immunity and are distinguished from other lymphocytes, such as B cells, by the presence of a T-cell receptor on the cell surface.

As used herein, the term “regulatory T cells” or “Treg cells” refers to cells that suppress, inhibit or prevent T cells activity. As used herein, Treg cells have the following phenotype at rest CD4+CD25+FoxP3+.

The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed”.

In some embodiments, the population of T cells is isolated from a subject to whom the genetically modified population of T cells is to be adoptively transferred. In some embodiments, a population of T cells of the present invention are obtained by isolating a population of T-cells from a subject, and by subsequently proceeding with FoxP3 gene transfer ex vivo with the viral vector of the present invention and subsequent immunotherapy of the subject by adoptive transfer of the transduced T cells. Alternatively, the population of T cells is isolated from a different subject, such that it is allogeneic. In some embodiments, the population of T cells is isolated from a donor subject.

Typically, the population of Treg cells is prepared as described in the EXAMPLE. The population of T cells is preactivated in an appropriate culture medium that contains an amount of a recombinant human IL-2 in the presence of anti-CD3/CD28 microbeads. T cells are then infected with the vector of the present invention. LNGFR+ transduced cells were purified by cell sorting. As used herein, the term “cell sorting” is used to refer to a method by which cells are mixed a binding partner (e.g., a fluorescently detectable antibody) in solution. According to the invention, any conventional cell sorting method may be used and typically involve use of anti-LNGFR antibodies. For instance, magnetic bead selection is suitable. Finally, the sorted T cells are expanded in presence of an amount of IL-2.

The population of Treg cells prepared as described above can be utilized in methods and compositions for adoptive immunotherapy in accordance with known techniques, or variations thereof that will be apparent to those skilled in the art based on the instant disclosure.

In particular, the population of Treg cells of the present invention is particularly suitable for the treatment of autoimmune diseases. The Treg cells as prepared by the method of the present invention may be administered for the purpose of suppressing autoimmune activity in a subject. As used herein, the term “autoimmunity” has its general meaning in the art and refers to the presence of a self-reactive immune response (e.g., auto-antibodies, self-reactive T-cells). Autoimmune diseases, disorders, or conditions arise from autoimmunity through damage or a pathologic state arising from an abnormal immune response of the body against substances and tissues normally present in the body. Damage or pathology as a result of autoimmunity can manifest as, among other things, damage to or destruction of tissues, altered organ growth, and/or altered organ function. Types of autoimmune diseases, disorders or conditions include type I diabetes, alopecia areata, vasculitis, temporal arteritis, rheumatoid arthritis, lupus, celiac disease, Sjogrens syndrome, polymyalgia rheumatica, and multiple sclerosis.

In particular, the Treg cells of the present invention are particularly suitable for the treatment of IPEX syndrome.

As used herein, the term “IPEX syndrome” has its general meaning in the art and a disease that results in most cases from mutations in FoxP3. IPEX syndrome usually develops during the first few days or weeks of life and affects exclusively boys. It manifests with the sequential appearance of the triad of enteropathy, autoimmune disease, and cutaneous involvement, but the clinical features and severity of the disease can vary considerably between individuals. Severe autoimmune enteropathy manifests with intractable secretory diarrhea leading to malabsorption, electrolyte disturbance and failure to thrive. Vomiting, ileus, gastritis or colitis can also be observed. Patients also present with autoimmune endocrinopathies, generally insulin-dependent diabetes mellitus (type 1 DM), but also thryroiditis leading to hypothyroidism or hyperthyroidism. Skin involvement consists of a generalized pruriginous eruption resembling eczema, psoriasis, and/or atopic or exfoliative dermatitis. Less frequently, alopecia or onychodystrophy can be observed. Patients may develop autoimmune cytopenias, thrombocytopenia, hemolytic anemia and neutropenia. Autoimmune involvement may also lead to pneumonitis, hepatitis, nephritis, myositis, splenomegaly and/or lymphadenopathy. Local or systemic infections (e.g. pneumonia, Staphylococcus aureus infections, candidiasis) may occur but seem to be due to loss of skin and gut barriers, immunosuppressive therapies, and poor nutrition rather than a primary immunodeficiency. IPEX syndrome is caused by mutations in the FOXP3 gene (Xp11.23). More than 20 mutations of FOXP3 are reported in IPEX, and the syndrome is lethal if untreated. Diagnosis is based on clinical examination, family history, and laboratory findings revealing autoimmune enteropathy (anti-enterocyte, harmonin and villin autoantibodies), type 1 DM (antibodies against insulin, pancreatic islet cells, or anti-glutamate decarboxylase), thyroiditis (anti-thyroglobulin and anti-microsome peroxidase antibodies) and cytopenia (anti-platelets and anti-neutrophils antibodies, positive Coombs test). Molecular genetic testing confirms the diagnosis.

Accordingly, a further object of the present invention relates to a method of treating an autoimmune disease (e.g. IPEX syndrome) in a subject in need thereof, comprising administering to the subject a therapeutically effective amount of a population of Treg cells of the present invention.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By a “therapeutically effective amount” is meant a sufficient amount of cells generated with the present invention for the treatment of the disease at a reasonable benefit/risk ratio applicable to any medical treatment. It will be understood that the total usage of these cells will be decided by the attending physicians within the scope of sound medical judgment. The specific therapeutically effective dose level for any particular subject will depend upon a variety of factors including the age, body weight, general health, sex and diet of the subject; the time of administration, route of administration, and survival rate of the cells employed; the duration of the treatment; drugs used in combination or coincidental with the administered cells; and like factors well known in the medical arts. For example, it is well known within the skill of the art to start doses of cells at levels lower than those required to achieve the desired therapeutic effect and to gradually increase the dosage until the desired effect is achieved.

In some embodiments, the Tregs cells are formulated by first harvesting them from their culture medium, and then washing and concentrating the cells in a medium and container system suitable for administration (a “pharmaceutically acceptable” carrier) in a treatment-effective amount. Typically, the population of Tregs cells of the present invention is administered to the subject in the form of pharmaceutical composition. The pharmaceutical composition may be produced by those of skill, employing accepted principles of treatment. Such principles are known in the art, and are set forth, for example, in Braunwald et al., eds., Harrison's Principles of Internal Medicine, 19th Ed., McGraw-Hill publisher, New York, N.Y. (2015), which is incorporated by reference herein. The pharmaceutical composition may be administered by any means that achieve their intended purpose. For example, administration may be by parenteral, subcutaneous, intravenous, intradermal, intramuscular, intraperitoneal, transdermal, or buccal routes. The pharmaceutical compositions may be administered parenterally by bolus injection or by gradual perfusion over time. The pharmaceutical compositions typically comprises suitable pharmaceutically acceptable carriers comprising excipients and auxiliaries which may facilitate processing of the active compounds into preparations which can be used pharmaceutically. The pharmaceutical compositions may contain from about 0.001 to about 99 percent, or from about 0.01 to about 95 percent of active compound(s), together with the excipient.

The invention will be further illustrated by the following figures and examples. However, these examples and figures should not be interpreted in any way as limiting the scope of the present invention.

### FIGURES

FIG. 1 depicts the different constructions tested by the inventors.

FIG. 2 depicts the flow cytometry analysis at Day 5.

### EXAMPLE

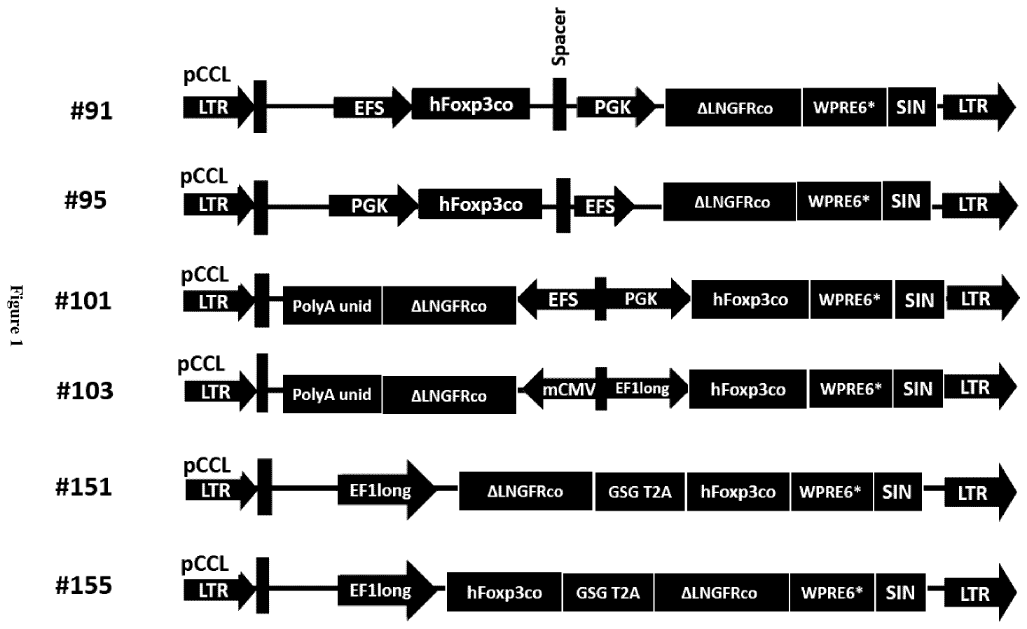

We compared 6 different lentiviral constructs according to 4 criteria (vector titers, level of transduction of human CD4+ T cells, level of expression of FOXP3 and ΔLNGFR genes, degree of correlation between both expression) (FIG. 1):
#91: unidirectional, EFS-FOXP3, PGK-ΔLNGFR
#95: unidirectional, PGK-FOXP3, EFS-ΔLNGFR
#101: bidirectional, ΔLNGFR-EFS,PGK-FOXP3
#103: bidirectional, ΔLNGFR-mCMV, EF1a-FOXP3
#151: bicistronic, EF1a-ΔLNGFR-T2A-FOXP3
#155: bicistronic, EF1a-FOXP3-T2A-ΔLNGFR

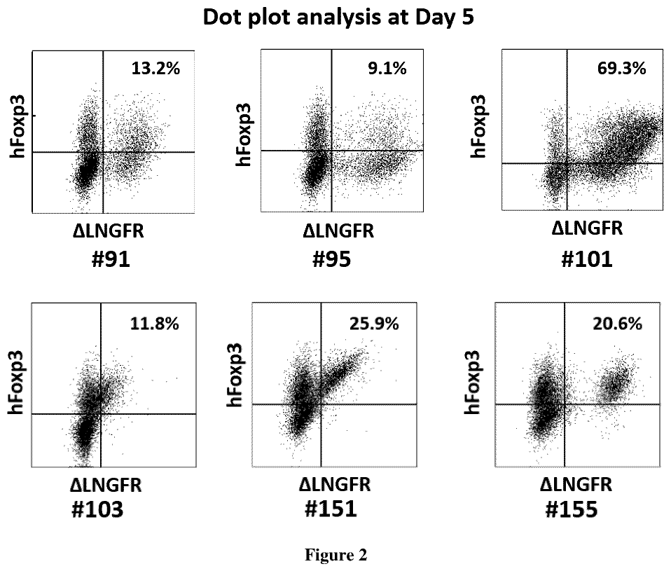

Table 1 below illustrates vector titer, transduction efficiency measured in vector copy number (VCN) per cell at day 12 of culture, and co-expression of FOXP3 and ΔLNGFR measured by flow cytometry indicated as % of CD4+ T cells at day 5. In some cases, ΔLNGFR+ cells were sorted at day 5, further cultured for 12 days and analyzed by flow cytometry at D12 (FIG. 2).

TABLE 1

| | | | % LNGFR + | % LNGFR + |
|---|---|---|---|---|
| | | VCN | FOXP3 + | FOXP3 + (D12) |
| Vector | Titer | (D12) | (D5) | after sorting at D5 |
| #91 | 1.49 × 10e9 | 4 | 13.2 | ND |
| #95 | ND | ND | 9.1 | ND |
| #101 | 4.1 × 10e9 | 4 | 69 | 88 |
| #103 | 1.3 × 10e9 | 0.45 | 11.8 | ND |
| #151 | 2.35 × 10e8 | 0.61 | 25.9 | ND |
| #155 | 6.36 × 10e7 | 0.31 | 20.6 | ND |

Constructs #151 and #155 were excluded because of low titers. #95 and #103 were excluded because of low levels of co-expression of FOXP3 and ΔLNGFR genes and low VCN for #103. #91 was excluded because of the low level of expression of FOXP3. To note, the bidirectional construct tested by Passerini and coll (Passerini et al., 2013) that we reproduce herein with the codon optimized version (#103) was not efficient in terms of correlation of expression of FOXP3 and ΔLNGFR genes. The only constructs that fulfilled the 4 criteria defined above is the bidirectional designs including forward hFOXP3co under the control of the PGK promoter and reverse ΔLNGFRco under the control of EFS promoter (#101, pCCL.ΔLNGFRco.EFS.PGK.hFOXP3co).

### REFERENCES

Throughout this application, various references describe the state of the art to which this invention pertains. The disclosures of these references are hereby incorporated by reference into the present disclosure.
Aarts-Riemens, T., M. E. Emmelot, L. F. Verdonck, and T. Mutis. 2008. Forced overexpression of either of the two common human Foxp3 isoforms can induce regulatory T cells from CD4(+)CD25(−) cells. Eur J Immunol 38:1381-1390.
Aiuti, A., R. Bacchetta, R. Seger, A. Villa, and M. Cavazzana-Calvo. 2012. Gene therapy for primary immunodeficiencies: Part 2. Curr Opin Immunol 24:585-591.
Aiuti, A., S. Vai, A. Mortellaro, G. Casorati, F. Ficara, G. Andolfi, G. Ferrari, A. Tabucchi, F. Carlucci, H. D. Ochs, L. D. Notarangelo, M. G. Roncarolo, and C. Bordignon. 2002. Immune reconstitution in ADA-SCID after PBL gene therapy and discontinuation of enzyme replacement. Nat Med 8:423-425.
Allan, S. E., A. N. Alstad, N. Merindol, N. K. Crellin, M. Amendola, R. Bacchetta, L. Naldini, M. G. Roncarolo, H. Soudeyns, and M. K. Levings. 2008. Generation of potent and stable human CD4+T regulatory cells by activation-independent expression of FOXP3. Mol Ther 16:194-202.
Barzaghi, F., L. Passerini, and R. Bacchetta. 2012. Immune dysregulation, polyendocrinopathy, enteropathy, x-linked syndrome: a paradigm of immunodeficiency with autoimmunity. Front Immunol 3:211.
Bennett, C. L., J. Christie, F. Ramsdell, M. E. Brunkow, P. J. Ferguson, L. Whitesell, T. E. Kelly, F. T. Saulsbury, P. F. Chance, and H. D. Ochs. 2001. The immune dysregulation, polyendocrinopathy, enteropathy, X-linked syndrome (IPEX) is caused by mutations of FOXP3. Nat Genet 27:20-21.
Blaese, R. M., K. W. Culver, A. D. Miller, C. S. Carter, T. Fleisher, M. Clerici, G. Shearer, L. Chang, Y. Chiang, P. Tolstoshev, J. J. Greenblatt, S. A. Rosenberg, H. Klein, M. Berger, C. A. Mullen, W. J. Ramsey, L. Muul, R. A. Morgan, and W. F. Anderson. 1995. T lymphocyte-directed gene therapy for ADA-SCID: initial trial results after 4 years. Science 270:475-480.
Bonino, C., G. Ferrari, S. Verzeletti, P. Servida, E. Zappone, L. Ruggieri, M. Ponzoni, S. Rossini, F. Mavilio, C. Traversari, C. Bordignon. 1997. HSV-TK Gene Transfer into Donor Lymphocytes for Control of Allogeneic Graft-Versus-Leukemia. Science 276: 1717-1724.
Bonini, C., M. K. Brenner, H. E. Heslop, and R. A. Morgan. 2011. Genetic modification of T cells. Biol Blood Marrow Transplant 17:S15-20.
Brunkow, M. E., E. W. Jeffery, K. A. Hjerrild, B. Paeper, L. B. Clark, S. A. Yasayko, J. E. Wilkinson, D. Galas, S. F. Ziegler, and F. Ramsdell. 2001. Disruption of a new forkhead/winged-helix protein, scurfin, results in the fatal lymphoproliferative disorder of the scurfy mouse. Nat Genet 27:68-73.
Ferraro, A., A. M. D′Alise, T. Raj, N. Asinovski, R. Phillips, A. Ergun, J. M. Replogle, A. Bernier, L. Laffel, B. E. Stranger, P. L. De Jager, D. Mathis, and C. Benoist. 2014. Interindividual variation in human T regulatory cells. Proc Natl Acad Sci USA 111:E1111-1120.
Fontenot, J. D., M. A. Gavin, and A. Y. Rudensky. 2003. Foxp3 programs the development and function of CD4+CD25+ regulatory T cells. Nat Immunol 4:330-336.
Fu, W., A. Ergun, T. Lu, J. A. Hill, S. Haxhinasto, M. S. Fassett, R. Gazit, S. Adoro, L. Glimcher, S. Chan, P. Kastner, D. Rossi, J. J. Collins, D. Mathis, and C. Benoist. 2012. A multiply redundant genetic switch ‘locks in’ the transcriptional signature of regulatory T cells. Nat Immunol 13:972-980.
Hori, S., T. Nomura, and S. Sakaguchi. 2003. Control of regulatory T cell development by the transcription factor Foxp3. Science 299:1057-1061.
Horino, S., Y. Sasahara, M. Sato, H. Niizuma, S. Kumaki, D. Abukawa, A. Sato, M. Imaizumi, H. Kanegane, Y. Kamachi, S. Sasaki, K. Terui, E. Ito, I. Kobayashi, T. Ariga, S. Tsuchiya, and S. Kure. 2014. Selective expansion of donor-derived regulatory T cells after allogeneic bone marrow transplantation in a patient with IPEX syndrome. Pediatr Transplant 18:E25-30.
Kalos, M., and C. H. June. 2013. Adoptive T cell transfer for cancer immunotherapy in the era of synthetic biology. Immunity 39:49-60.
Kasow, K. A., V. M. Morales-Tirado, D. Wichlan, S. A. Shurtleff, A. Abraham, D. A. Persons, and J. M. Riberdy. 2011. Therapeutic in vivo selection of thymic-derived natural T regulatory cells following non-myeloablative hematopoietic stem cell transplant for IPEX. Clin Immunol 141:169-176.
Khattri, R., T. Cox, S. A. Yasayko, and F. Ramsdell. 2003. An essential role for Scurfin in CD4+CD25+T regulatory cells. Nat Immunol 4:337-342.
Li, W., L. Wang, H. Katoh, R. Liu, P. Zheng, and Y. Liu. 2011. Identification of a tumor suppressor relay between the FOXP3 and the Hippo pathways in breast and prostate cancers. Cancer Res 71:2162-2171.
Mottet, C., H. H. Uhlig, and F. Powrie. 2003. Cutting edge: cure of colitis by CD4+CD25+ regulatory T cells. J Immunol 170:3939-3943.
Muul, L. M., L. M. Tuschong, S. L. Soenen, G. J. Jagadeesh, W. J. Ramsey, Z. Long, C. S. Carter, E. K. Garabedian, M. Alleyne, M. Brown, W. Bernstein, S. H. Schurman, T. A. Fleisher, S. F. Leitman, C. E. Dunbar, R. M. Blaese, and F. Candotti. 2003. Persistence and expression of the adenosine deaminase gene for 12 years and immune reaction to gene transfer components: long-term results of the first clinical gene therapy trial. Blood 101:2563-2569.
Passerini, L., E. Rossi Mel, C. Sartirana, G. Fousteri, A. Bondanza, L. Naldini, M. G. Roncarolo, and R. Bacchetta. 2013. CD4(+) T cells from IPEX patients convert into functional and stable regulatory T cells by FOXP3 gene transfer. Sci Transl Med 5:215ra174.
Provasi, E., P. Genovese, A. Lombardo, Z. Magnani, P. Q. Liu, A. Reik, V. Chu, D. E. Paschon, L. Zhang, J. Kuball, B. Camisa, A. Bondanza, G. Casorati, M. Ponzoni, F. Ciceri, C. Bordignon, P. D. Greenberg, M. C. Holmes, P. D. Gregory, L. Naldini, and C. Bonini. 2012. Editing T cell specificity towards leukemia by zinc finger nucleases and lentiviral gene transfer. Nat Med 18:807-815.
Sawant, D. V., and D. A. Vignali. 2014. Once a Treg, always a Treg? Immunol Rev 259:173-191.
Seidel, M. G., G. Fritsch, T. Lion, B. Jurgens, A. Heitger, R. Bacchetta, A. Lawitschka, C. Peters, H. Gadner, and S. Matthes-Martin. 2009. Selective engraftment of donor CD4+25high FOXP3-positive T cells in IPEX syndrome after nonmyeloablative hematopoietic stem cell transplantation. Blood 113:5689-5691.
Tang, Q., K. J. Henriksen, M. Bi, E. B. Finger, G. Szot, J. Ye, E. L. Masteller, H. McDevitt, M. Bonyhadi, and J. A. Bluestone. 2004. In vitro-expanded antigen-specific regulatory T cells suppress autoimmune diabetes. J Exp Med 199:1455-1465.
Thornton, A. M., and E. M. Shevach. 1998. CD4+CD25+ immunoregulatory T cells suppress polyclonal T cell activation in vitro by inhibiting interleukin 2 production. J Exp Med 188:287-296.
Trzonkowski, P., M. Bieniaszewska, J. Juscinska, A. Dobyszuk, A. Krzystyniak, N. Marek, J. Mysliwska, and A. Hellmann. 2009. First-in-man clinical results of the treatment of patients with graft versus host disease with human ex vivo expanded CD4+CD25+CD127-T regulatory cells. Clin Immunol 133:22-26.
Wieckiewicz, J., R. Goto, and K. J. Wood. 2010. T regulatory cells and the control of alloimmunity: from characterisation to clinical application. Curr Opin Immunol 22:662-668.
Wildin, R. S., F. Ramsdell, J. Peake, F. Faravelli, J. L. Casanova, N. Buist, E. Levy-Lahad, M. Mazzella, O. Goulet, L. Perroni, F. D. Bricarelli, G. Byrne, M. McEuen, S. Proll, M. Appleby, and M. E. Brunkow. 2001. X-linked neonatal diabetes mellitus, enteropathy and endocrinopathy syndrome is the human equivalent of mouse scurfy. Nat Genet 27:18-20.
Yagi, H., T. Nomura, K. Nakamura, S. Yamazaki, T. Kitawaki, S. Hori, M. Maeda, M. Onodera, T. Uchiyama, S. Fujii, and S. Sakaguchi. 2004. Crucial role of FOXP3 in the development and function of human CD25+CD4+ regulatory T cells. Int Immunol 16:1643-1656.

1. A recombinant nucleic acid molecule comprising a bidirectional EFS-PGK promoter operably linked to a first transgene in one direction and to a second transgene in the opposite direction, wherein the bidirectional EFS-PGK promoter comprises an EFS portion derived from the EFS promoter and a PGK portion derived from the PGK promoter, wherein the first transgene is under the control of the EFS portion of the bidirectional promoter and encodes a protein that is not constitutively expressed by a T cell and the second transgene is under the control of the PGK portion of the bidirectional promoter and encodes a transcription factor.

2. The recombinant nucleic acid molecule of claim 1 wherein the EFS portion comprises a nucleic sequence having at least 80% of identity with the nucleic acid sequence as set forth in SEQ ID NO:3.

3. The recombinant nucleic acid molecule of claim 1 wherein the PGK portion comprises a nucleic sequence having at least 80% of identity with the nucleic acid sequence as set forth in SEQ ID NO:2.

4. The recombinant nucleic acid molecule of claim 1 wherein the EFS portion and the PGK portion are separated by a spacer sequence.

5. The recombinant nucleic acid molecule of claim 4 wherein the spacer sequence comprises a nucleic sequence having at least 80% of identity with the nucleic acid sequence as set forth in SEQ ID NO:4.

6. The recombinant nucleic acid molecule of claim 1 wherein the bidirectional promoter comprises a nucleic acid sequence having at least 80% of identity with the sequence as set forth in SEQ ID NO:5.

7. The recombinant nucleic acid molecule of claim 1 wherein the sequences of the transgenes are codon-optimized.

8. The recombinant nucleic acid molecule of claim 1 wherein the first transgene that is under the control of the EFS portion of the bidirectional promoter encodes for a low-affinity nerve growth factor receptor (LNGFR).

9. The recombinant nucleic acid molecule of claim 1 wherein the second transgene that is under the control of the PGK portion of the bidirectional promoter encodes for FoxP3.

10. The recombinant nucleic acid molecule of claim 1 which comprises:
i) a first nucleic acid sequence having at least 80% of identity with the nucleic acid sequence as set forth in SEQ ID: 8
ii) a second nucleic acid sequence having at least 80% of identity with the nucleic acid sequence acid sequence as set forth in SEQ ID NO:5 and
iii) a third nucleic acid sequence having at least 80% of identity with the nucleic acid sequence as set forth in SEQ ID NO:7.

11. The recombinant acid molecule of claim 1 which comprises a nucleic acid sequence having at least 80% of identity with the nucleic acid sequence as set forth in SEQ ID NO:11.

12. A lentiviral vector which comprises the recombinant acid molecule of claim 1.

13. (canceled)

14. A method of producing a population of Treg cells, comprising, transfecting or transducing a population of T cells in vitro or ex vivo with the lentiviral vector of claim 12.

15. A population of Treg cells obtainable by the method of claim 14.

16. A method of treating an autoimmune disease in a subject in need thereof comprising administering to the subject a therapeutically effective amount of the population of Treg cells of claim 15.

17. The method of claim 16 wherein the autoimmune disease is IPEX syndrome.

18. A nucleic acid sequence as set forth in SEQ ID NO:7.

19. The method of claim 14, wherein the population of Treg cells that express LNGFR at their cell surface.

20. The recombinant nucleic acid molecule of claim 8, wherein the LNGFR is a low-affinity nerve growth factor receptor truncated of its intracytoplasmic part (ΔLNGFR).
